(19) United States
(12) Patent Application Publication
(10) Pub. No.: US 2005/0165075 A1
(43) Pub. Date: Jul. 28, 2005

(54) Novel amorphous form of valsartan

(75) Inventors: Reddy Bandi Parthasaradhi, Hyderabad (IN); Reddy Kura Rathnakar, Hyderabad (IN); Reddy Rapolu Raji, Hyderabad (IN); Reddy Dasari Muralidhara, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 10/509,495

(22) PCT Filed: Mar. 31, 2003
(86) PCT No.: PCT/IN03/00096

(51) Int. Cl. A61K 31/4184; C07D 43/02

(52) U.S. Cl. 514/381; 548/253

(57) ABSTRACT

The present invention relates to a novel amorphous form of valsartan, to a process for its preparation and to a pharmaceutical composition containing it.

### FIELD OF THE INVENTION

The present invention relates to a novel amorphous form of valsartan, to a process for its preparation and to a pharmaceutical composition containing it.

### BACKGROUND OF THE INVENTION

Valsartan of formula (1): 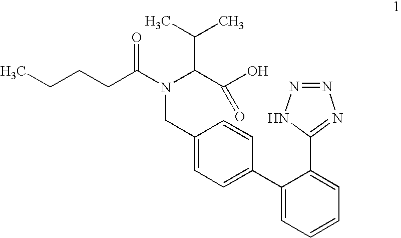
or N-(1-Oxopentyl)-N-[[2′-(1H-tetrazol-5-yl)[1,1′-biphenyl]-4-yl]methyl]-L-valine, is an antihypertensive agent and its therapeutic uses are disclosed in U.S. Pat. No. 5,399,578. No polymorphs of valsartan are reported in the literature.

We discovered a sufficiently stable amorphous form of valsartan, which is found to be suitable for pharmaceutical composition.

The object of the present invention is to provide a novel stable amorphous form of valsartan, process the preparing it and a pharmaceutical composition containing it.

### DETAILED DESCRIPTION OF THE INVENTION

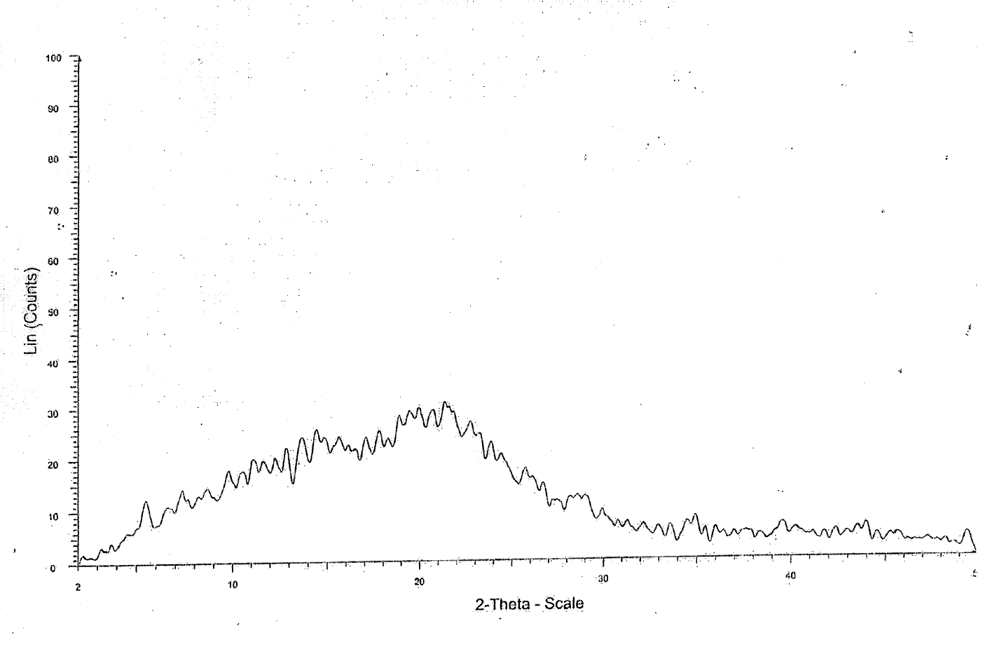

The present invention provides a novel amorphous form of valsartan (hereinafter referred to as amorphous valsartan). The amorphous valsartan is characterized by having broad x-ray diffraction spectrum as in FIG. 1.

A further aspect of the present invention provides a process for the preparation of amorphous valsartan. Amorphous valsartan is prepared by dissolving valsartan in an alcohol or a mixture of alcohols. The alcohol is selected from the group consisting of methanol, ethanol, isopropyl alcohol, tert-butyl alcohol and n-butyl alcohol. The solvent may be removed from the solution by vacuum drying or spray drying.

A further aspect of the present invention provides a pharmaceutical composition comprising amorphous valsartan and a pharmaceutically acceptable carrier.

### BRIEF DESCRIPTION OF THE DRAWINGS

FIG. 1 is a x-ray powder diffraction spectrum of amorphous valsartan.
x-Ray powder diffraction spectrum was measured on a Siemens D5000 x-ray powder diffractometer having a copper-Kα radiation.

The following examples further illustrate the invention.

### EXAMPLE 1

Valsartan (10 gm), (obtained by the process described in example-16 of U.S. Pat. No. 5,399,578) is dissolved in methanol (50 ml). The solution is subjected to vacuum drying at about 40° C. for 10 hours to give 9.8 gm of amorphous valsartan.

### EXAMPLE 2

Example 1 is repeated by subjecting the solution to spray drying instead of vacuum drying to give amorphous valsartan.

### EXAMPLE 3

Valsartan (10 gm), (obtained by the process described in example-16 of U.S. Pat. No. 5,399,578) is dissolved in ethanol (60 ml). The solution is subjected to vacuum drying at about 45° C. for 12 hours to give 9.7 gm of amorphous valsartan.

### EXAMPLE 4

Example 3 is repeated by subjecting the solution to spray drying instead of vacuum drying to give amorphous valsartan.

### EXAMPLE 5

Valsartan (10 gm) is dissolved in isopropyl alcohol (70 ml). The solution is subjected to vacuum drying at about 45° C. for 15 hours to give 9.9 gm of amorphous valsartan.

1. Amorphous valsartan characterized by an x-ray powder diffraction spectrum as in FIG. 1.

2. The process for preparation of amorphous valsartan of claim 1, which comprises:
a) dissolving valsartan in an alcohol or a mixture of alcohols;
b) removing the solvents from the solution formed in step (a) either by vacuum drying or by spray drying;
wherein the alcohol is selected from the group consisting of methanol, ethanol isopropyl alcohol, tert-butyl alcohol and n-butyl alcohol.

3. The process according to claim 3, wherein the solvent is removed by vacuum drying.

4. The process according to claim 3, wherein the solvent is removed by spray drying.

5. A pharmaceutical composition comprising amorphous valsartan and a pharmaceutically acceptable carrier.
